(19) United States
(12) Patent Application Publication
(10) Pub. No.: US 2007/0213835 A1
(43) Pub. Date: Sep. 13, 2007

(54) PLASTIC IMPLANT IMPREGNATED WITH AN ANTIBIOTIC

(76) Inventors: Markus Wimmer, Chicago, IL (US); Matthias Honl, Klagenfurt (DE)

Correspondence Address: Edward P. Gamson, Suite 2200, 120 S. Riverside Plaza, Chicago, IL, 60606-3945, US

(21) Appl. No.: 11/556,022

(22) Filed: Nov. 2, 2006

Related U.S. Application Data
(60) Provisional application No. 60/733,749, filed on Nov. 4, 2005.

(51) Int. Cl. A61F 2/28

(52) U.S. Cl. 623/23.58; 424/424

(57) ABSTRACT

A plastic device containing an effective amount of an antibiotic to minimize bacterial and/or fungal infection at the site of implantation is disclosed.

### CROSS-REFERENCE TO RELATED APPLICATION

This application claims priority from Ser. No. 60/733,749 filed on Nov. 04, 2005, whose disclosures are incorporated by reference.

### TECHNICAL FIELD

The present invention relates to an implant containing an antibiotic for reducing implant-related infection in vivo. In particular, a plastic portion of an implant is impregnated with one or both of an antibacterial agent such as gentamycin and an antifungal agent such as amphotericin B. These additives in the plastic improve the longevity of the implant in vivo by reducing the amount of bacterial growth at the implant site.

### BACKGROUND

There are approximately 200,000 hip replacement surgeries each year in the U.S. alone and about 800,000 worldwide. The number of knee replacement surgeries is approximately double this amount. In the future, the number of hip and knee replacement surgeries will continue to grow exponentially as the population ages.

Hip replacement surgery entails surgical intervention of two parts of the hip joint, the acetabulum (a cup-shaped bone in the pelvis) and the femoral head (the ball-shaped end of the thigh bone), then replacing each with smooth artificial surfaces. Exemplary artificial surfaces are high-density plastic, metal, or ceramic materials.

Total hip replacements are most often performed for severe painful arthritic conditions but sometimes are performed for other problems such as deformities, fractures, tumors, or aseptic necrosis of the bone. In the majority of cases, hip replacement surgery is very successful in relieving pain, restoring function, and markedly improving the quality of life for patients with hip disease. The caveat is that the average life of a prosthetic implant is only about ten years. This relatively short functional existence is due to problems with decomposition (wear) of the prosthetic's fabrication materials and loosening of the joint due to osteolysis (bone loss) and other complications.

Wear debris and the resulting osteolysis from inflammatory cellular responses to wear debris are the most significant factors contributing to failure of hip replacements. Osteolysis, when undetected and untreated, results in massive bone loss and implant failure.

Joint revision (replacement) procedures can be more difficult than the original surgery due to diseased, damaged, and decomposed bone in the area of the former prosthesis. Often there is little original natural bone remaining to attach a new prosthesis to after the damaged prosthesis is removed. The magnitude of this problem is significant. For example, in 1996, total hip revision rates were about one third of the primary hip replacements. The function and long term survival of revision total hip arthroplasty is generally inferior to primary hip arthroplasty and leads to a worsened quality of life for many patients.

The amount of wear in a hip prosthesis is dependent upon many factors, including the materials used in fabrication, osteolysis from inflammatory cellular responses, bacterial and/or fungal infection at the implant site the weight of the patient, the age of the patient, and the patient's activity level, to name just a few. As mentioned above, materials commonly utilized in fabrication of hip prosthetics include plastic (ultra high molecular weight polyethylene, UHMWPE), metal (titanium or cobalt-chromium alloy), or alumina and zirconia ceramics.

The amount of wear for polymeric materials is in the region of 50-100 mm³ per year for UHMWPE and 10 mm³ (or smaller) per year for the newest cross-linked UHMWPE The wear debris comes from the grinding of the metal against the polymer as the patient moves the prosthetic hip. Metal on metal and ceramic on ceramic implants sometimes have less wear debris than plastic implants; however they come with other risks, including suffering adverse biological effects to increased metal ions in the body, experiencing chipping of ceramic components, and risking fracture of the implant. Also, with metal and ceramic implants there are additional hurdles to overcome in fabrication such as the difficulty in obtaining conforming surfaces and consistently correct clearances. Lastly, wear debris is often associated with bacterial and/or fungal infection in the bone causing failure of the prosthesis.

The contamination and colonization of the polyethylene liner of a total joint prosthesis with bacteria is found in about 70% of all cases. Of these cases, approximately 65% of the infections are caused by aerobic gram-positive cocci including Staphylococcus aureus, coagulase-negative staphylococci, streptococci, and enterococci. Recently, it has been shown that certain bacteria colonize and produce a slime layer or glycocalix. Many species of S. aureus and S. epidermidis, which seem to be prevalent at the site of implant infection, are slime producers. The bacteria's ability to produce a slime layer permits the bacteria to exist within a biofilm of glycocalix that allows it to adhere to and survive on synthetic surfaces such as polyethylene.

Bacteria that exist within a biofilm are at least 500 times more resistant to antibiotics than the free-floating forms. They are also relatively isolated from host defense mechanisms. Bacterial biofilms require a certain minimum time to form after the inoculation of the infecting organism. In vitro evidence has suggested that infections can be eradicated with antibiotics while the inoculated organism is still free form but not after a biofilm has formed. Thus, it is essential that the infection be caught quickly or, as in the present invention, be precluded from forming at all.

Prosthetic joint infection can be classified into several major types: during surgery, several months after prosthesis implantation, and late chronic infection diagnosed months to years after prosthesis implantation.

The earliest infections sometimes can be lessened with parenteral antimicrobial therapy or chronic oral antimicrobial suppression. Later found infections sometimes can be lessened by surgical removal of damaged tissue/bone followed by parenteral antimicrobial therapy. Late chronic infections sometimes can be lessened by the surgical removal of the prosthesis followed by delayed reimplantation of a new prosthesis. However, amputation of the appendage below the bone, as in infected knee prosthesis cases, may be required because of problems such as poor bone stock, multiple prior infections, and inability to undergo further surgery. Death may occur in some cases due to the spread of infection and associated complications.

Thus, infection at the site of prosthetic implantation is a serious problem. Administering prophylactic antibiotics in the perioperative period is currently standard of care. However, in many cases, this practice is not effective due to the presence of the bacterial or fungal slime layer at the prosthesis implant site. The use of antibiotic-impregnated bone cement used to affix the prosthesis to the bone stock is another method that may lessen postoperative infection. However, the inclusion of an antibiotic into the cement mixture alters the mechanical properties of the cement, specifically reducing the fatigue of the material, which determines its resistance to crack formation and the long-term in vivo structural integrity of the cement mantle. Also, in physical terms, there is very little bone cement present to set the prosthesis, thus very little antibiotic is truly available at the implantation site. Usually, there is not an effective amount of antibiotic available in these cases.

Thus, there is an urgent need for a means to protect an implant device such as a prosthesis and its implant site from microbial infection and to increase the longevity of the implant by minimizing the amount of wear due to infection at the site. The invention disclosed hereinafter provides one such anti-microbial protection means.

### BRIEF SUMMARY OF THE INVENTION

One aspect of the present invention contemplates a synthetic plastic-containing implant device such as a prosthesis that contains an effective amount of an antibiotic (anti-microbial) compound substantially homogeneously distributed throughout the plastic. Preferably, the antibiotic compound is an antibacterial compound or an antifungal compound or a mixture of both. More preferably, the antibiotic is present in an amount of about 0.01 to about 5 percent by weight of the plastic portion of the implant, such as polyethylene. Preferably, the synthetic plastic is ultra high molecular weight polyethylene or polyetheretherketon.

The antibiotic is preferably an antibacterial compound selected from the group consisting of an aminoglycoside, a lincosamide, a glycopeptide and mixtures thereof. A preferred that the antifungal compound is selected from the group consisting of a polyene, an azole, an allylamine, a morpholine, a glucan synthesis inhibitor, a systemic agent, an antimetabolite and mixtures thereof. More preferably, the antibiotic is gentamycin and the antifungal is amphotericin B.

Another aspect of the invention is a method for reducing wear in a prosthesis comprised of plastic that is implanted in a mammal. In accordance with that method, an effective amount of an antibiotic compound is substantially homogeneously distributed throughout the plastic implant.

The present invention has several benefits and advantages.

One benefit is that it provides a plastic prosthetic implant that contains an antibiotic to prevent the colonization of bacteria at the implant site that leads to loosening and failure of the implant.

An advantage of the invention is that it can be more effective than mere use of an anti-microbial agent in bone cement because the prosthesis offers a greater dosage of the antibiotic at the implant site because more antibiotic can be physically loaded into the implant and is available at the larger surface area of the implant versus the smaller amount and surface area antibiotic-laden bone cement.

Another benefit of the invention is that its use provides lessened wear to the prosthetic device after it is implanted in the mammalian host.

Still further benefits and advantages of the invention will be apparent to the worker of ordinary skill from the disclosure that follows.

### DETAILED DESCRIPTION OF THE INVENTION

The present invention contemplates a plastic-containing implant device impregnated with an effective amount of an antibiotic (anti-microbial) compound substantially homogeneously distributed throughout the synthetic plastic. The implant device (implant) is intended for use in a mammal, preferably a human. The synthetic plastic is preferably ultra high molecular weight polyethylene or polyetheretherketon, which have been shown in clinical studies to generate fewer problems as compared to metal or ceramic implants.

Preferably, the antibiotic compound is an antibacterial agent or antifungal agent or a mixture of the two, but can be any compound or chemical element or mixtures thereof with antibacterial or antifungal properties at the concentration used in an implant. In one aspect, the antibiotic is an antibacterial compound selected from the group consisting of an aminoglycoside, a lincosamide, a glycopeptide and mixtures thereof. More preferably, the antibiotic is one or more of gentamycin, clindamycin, or vancomycin.

A preferred antifungal compound is selected from the group consisting of a polyene, an azole, an allylamine, a morpholine, a glucan synthesis inhibitor, a systemic agent, an antimetabolite and mixtures thereof. More preferably, the antifungal agent is amphotericin B.

For most antibiotics, an effective amount as used herein is an amount of about 0.01 to about 5 percent by weight, and more preferably about 0.1 to about 3 percent by weight, of plastic such as polyethylene. In another embodiment, the antibiotic such as gentamycin is present in an amount of about 0.0225 g/ml per one ml of plastic such as polyethylene. The words “antibiotic” and “antimicrobial” in their various grammatical forms are used interchangeably herein.

Substantially any bone or joint can be fabricated as a prosthesis as described above, including maxofacial, shoulder, elbow, ankle, finger, toe, wrist, neck, rib, spinal, cervical, and the like. However, a preferred prosthesis is either a hip joint or a knee joint. Although a contemplated prosthesis can be implanted in any mammal, such as in a mouse, rat, rabbit, cat, dog, sheep, bovine, or horse, the mammal is preferably a human patient.

To prepare a contemplated prosthesis, one need only mix the precursor plastic, e.g., beads or flakes or the like, with the antibiotic compound or compounds. The admixed materials are brought into a molten stage and the prosthesis is cast (molded) or extruded as desired, and then machined or otherwise handled as is usually done for a plastic portion of a prosthesis.

A further aspect of the invention is a method for reducing wear in a prosthesis comprised at least in part of plastic that is implanted in a mammal host. In accordance with that method, an effective amount of an antibiotic compound as discussed previously is substantially homogeneously distributed throughout the plastic.

### EXAMPLE

### Example 1

### In Vitro Wear Test of a Polyethylene Prosthesis With Exogenously Supplied Antibiotic and an Antifungal Agent

In this example, several small pins of ultra high molecular weight polyethylene were manufactured and their physical properties tested in a standard wear testing apparatus to which an antibacterial agent and an antifungal agent were added to the usually used lubricating liquid.

Simulators designed to mimic the motions and loads of hip and knee joints are widely used to evaluate the polyethylene component wear in total joint prostheses. A total of twelve pins (10 mm in diameter) were manufactured out of GUR™415, ultra-high-molecular-weight polyethylene (UHMWPE) (Hoechst Celanese, Houston, Tex.). A constant load of 298 N (3.8 MPa) was applied on every pin using a 6-station pin-on-disc wear machine (Orthopod™, AMTI). Each pin articulated against polished CoCr (with a 5×5 mm square motion) and was submerged in 15 ml of new born calf serum diluted to a protein content of 30 g/L. To prevent bacterial and fungal contamination, 1 g/kg of gentamycin and 11 g/kg fungizone were added in trial 1 (6 pins). No additives were used in trial 2. Both trials were performed up to 1 million cycles, while trial 2 was continued for another million cycles switching lubricants (with and without additives) in-between measurement points.

Every 250,000 cycles, the equivalent of 3 days, the pins were dismounted, ultrasonically cleaned and weighed with a high precision balance. Determined weight loss was corrected for soak (3 non-loaded specimens per trial). In addition, fluid samples were taken from the lubricant. The protein content of the samples was determined using micro BCA™ protein assay. Bacterial and/or fungal contamination was investigated using agar culture.

Wear rates with antimicrobial additives in the lubricant were 6.5-fold smaller than without (p<0.001). Frictional forces were also greater in the absence of the antibiotics than when they were present in the lubricant. Soak controls were not affected. With antibiotic additives in place, during the second million cycles of trial 2, the wear rates diminished greatly and did not differ from those of trial 1 (p>0.1). Most of the samples of trial 1 were contaminated with bacillus, micrococcus (bacteria) and penicillium (fungus). Interestingly, the protein content of samples without contamination was lower than those with (p<0.004). However, using a linear regression model only 25% of the variance could be explained by protein content.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A device for implantation in a mammal that comprises a synthetic plastic that contains an effective amount of an antibiotic compound substantially homogeneously distributed throughout the synthetic plastic.

2. The device according to claim 1 wherein the antibiotic compound is an antibacterial compound or antifungal compound.

3. The device according to claim 1 wherein the antibiotic is present in an amount of about 0.01to about 5 percent by weight of the plastic.

4. A device for implantation in a mammal comprising synthetic plastic that contains about 0.01 to about 5 percent by weight of the plastic of an antibiotic that is an antifungal or antibacterial compound or both and antibacterial and an antifungal compound substantially homogeneously distributed through out the synthetic plastic.

5. The device according to claim 4 wherein the antibiotic is an antibacterial compound selected from the group consisting of an aminoglycoside, a lincosamide, a glycopeptide and mixtures thereof.

6. The device according to claim 4 wherein the antibiotic is present in an amount of about 0.1 to about 3 percent by weight of the plastic.

7. The device according to claim 4 wherein the synthetic plastic is ultra high molecular weight polyethylene or polyetheretherketon.

8. The deviceaccording to claim 4 wherein the antibiotic is an antifungal compound that is selected from the group consisting of a polyene, an azole, an allylamine, a morpholine, a glucan synthesis inhibitor, a systemic agent, an antimetabolite and mixtures thereof.

9. A device for implantation in a mammal comprising an ultra high molecular weight polyethylene that contains about 0.1 to about 3 percent by weight of the plastic of an antibiotic substantially homogeneously distributed through out the synthetic plastic, wherein said antibiotic is one or more of an antibacterial compound selected from the group consisting of a aminoglycoside, a lincosamide, a glycopeptide and mixtures thereof and an antifungal compound selected from the group consisting of a polyene, an azole, an allylamine, a morpholine, a glucan synthesis inhibitor, a systemic agent, an antimetabolite and mixtures thereof.

10. The device according to claim 9 wherein the antibiotic is gentamycin.

11. The device according to claim 9 wherein the antibiotic is clindamycin.

12. The device according to claim 9 wherein the antibiotic is vancomycin.

13. The device according to claim 9 wherein the ultra high molecular weight polyethylene is cross-linked.

14. The device according to claim 9 that is a hip joint.

15. The device according to claim 9 that is a knee joint.

16. The device according to claim 9 wherein said mammal is a human patient.

17. A device for implantation in a mammal that comprises an ultra high molecular weight polyethylene that contains an effective amount of an antifungal compound substantially homogeneously distributed throughout the synthetic plastic.

18. The device according to claim 17 wherein the antifungal compound is present in an amount of 0.1 to about 3 percent by weight of the polyethylene.

19. The device according to claim 17 wherein the antifungal is amphotericin B or a derivative thereof.

20. The device according to claim 17 that is a hip joint.

21. The device according to claim 17 that is a knee joint.

22. The device according to claim 17 wherein said mammal is a human patient.

23. The device according to claim 17 wherein the ultra high molecular weight polyethylene is cross-linked.

24. A method of reducing wear of a plastic-containing prosthesis implanted in a mammal that comprises substantially homogeneously distributing an effective amount of an antibiotic compound throughout the plastic.
